(19) United States
(12) Patent Application Publication
(10) Pub. No.: US 2011/0171326 A1
(43) Pub. Date: Jul. 14, 2011

(54) Pharmaceutical material for psoriasis treatment

(76) Inventors: Chao-Ho Liu, Taichung (TW); Meng-Han Liu, Taichung (TW); Jia-Ying Lin, Taichung (TW)

(21) Appl. No.: 12/684,116

(22) Filed: Jan. 8, 2010

(51) Int. Cl. A61K 36/24; A61K 36/00; A61P 17/06

(52) U.S. Cl. 424/725

(57) ABSTRACT

One embodiment of a pharmaceutical material for psoriasis treatment includes a liquid preparation by extracting herbs selected from the group consisting of Artemisia capillaris, Plumeria acutifolia, and Melaleuca alternifolia. Artemisia capillaris is about 5 to about 21 weight % of the liquid preparation, Plumeria acutifolia is about 65 to about 90 weight % of the liquid preparation, and Melaleuca alternifolia is about 4 to about 16 weight % of the liquid preparation respectively.

### BACKGROUND OF THE INVENTION

1. Field of Invention

The invention relates to the treatment of psoriasis and more particularly to an improved pharmaceutical material prepared from herbs for the treatment of psoriasis.

2. Description of Related Art

Psoriasis is a chronic, non-contagious disease that affects mainly the skin. It typically causes red, scaly patches to appear on the skin. The scaly patches caused by psoriasis, called psoriatic plaques, are areas of inflammation and excessive skin production. Fingernails and toenails are frequently affected.

Treatments of psoriasis are well known. For example, U.S. Pat. No. 4,826,677 discloses a pharmaceutical material for topical treating of psoriasis. The disadvantages of some topical agents include, but not limited to, normal skin irritation, time consuming, and being not suitable for use for long periods of time. Thus, continuing improvements in the exploitation of pharmaceutical material for psoriasis treatment are constantly being sought.

### SUMMARY OF THE INVENTION

It is therefore one object of the invention to provide a pharmaceutical material for treating of psoriasis comprising a liquid preparation by extracting herbs selected from the group consisting of Artemisia capillaris, Plumeria acutifolia, and Melaleuca alternifolia.

It is another object of the invention to provide a pharmaceutical material for treating of psoriasis comprising a dustlike preparation by powdering herbs selected from the group consisting of Artemisia capillaris, Plumeria acutifolia, and Melaleuca alternifolia.

In one aspect of the invention Artemisia capillaris is about 5 to about 21 weight % of the preparation, Plumeria acutifolia is about 65 to about 90 weight % of the preparation, and Melaleuca alternifolia is about 4 to about 16 weight % of the preparation respectively.

### DETAILED DESCRIPTION OF THE INVENTION

A pharmaceutical material for psoriasis treatment in accordance with a first preferred embodiment of the invention comprises a liquid preparation by extracting herbs selected from the group consisting of Artemisia capillaris, Plumeria acutifolia, and Melaleuca alternifolia.

Preferably, Artemisia capillaris is about 5 to about 21 weight % of the preparation, Plumeria acutifolia is about 65 to about 90 weight % of the preparation, and Melaleuca alternifolia is about 4 to about 16 weight % of the preparation respectively.

A pharmaceutical material for psoriasis treatment in accordance with a second preferred embodiment of the invention comprises a dustlike preparation by powdering herbs selected from the group consisting of Artemisia capillaris, Plumeria acutifolia, and Melaleuca alternifolia.

Preferably, Artemisia capillaris is about 5 to about 21 weight % of the preparation, Plumeria acutifolia is about 65 to about 90 weight % of the preparation, and Melaleuca alternifolia is about 4 to about 16 weight % of the preparation respectively.

In each of the first and second preferred embodiments, the preparation can be applied to the skin.

In each of the first and second preferred embodiments, the preparation can be produced in the form of, in addition to above liquid and dustlike form, ointment, adhering film, solution, moisturizer, jelly, cream, lotion, bath oil, etc.

While the invention herein disclosed has been described by means of specific embodiments, numerous modifications and variations could be made thereto by those skilled in the art without departing from the scope and spirit of the invention set forth in the claims.

What is claimed is:

1. A pharmaceutical material for treating of psoriasis comprising a liquid preparation by extracting herbs selected from the group consisting of Artemisia capillaris, Plumeria acutifolia, and Melaleuca alternifolia.

2. The pharmaceutical material of claim 1, wherein Artemisia capillaris is about 5 to about 21 weight % of the preparation, Plumeria acutifolia is about 65 to about 90 weight % of the preparation, and Melaleuca alternifolia is about 4 to about 16 weight % of the preparation respectively.

3. A pharmaceutical material for treating of psoriasis comprising a dustlike preparation by powdering herbs selected from the group consisting of Artemisia capillaris, Plumeria acutifolia, and Melaleuca alternifolia.

4. The pharmaceutical material of claim 3, wherein Artemisia capillaris is about 5 to about 21 weight % of the preparation, Plumeria acutifolia is about 65 to about 90 weight % of the preparation, and Melaleuca alternifolia is about 4 to about 16 weight % of the preparation respectively.
